(19) United States
(12) Patent Application Publication
(10) Pub. No.: US 2004/0115701 A1
(43) Pub. Date: Jun. 17, 2004

(54) Method for risk assessment for polygenic disorders

(76) Inventors: David E. Comings, Monrovia, CA (US); James P. MacMurray, Claremont, CA (US)

Correspondence Address: PERKINS COIE LLP, POST OFFICE BOX 1208, SEATTLE, WA, 98111-1208, US

(21) Appl. No.: 10/654,642

(22) Filed: Sep. 2, 2003

Related U.S. Application Data
(60) Provisional application No. 60/407,341, filed on Aug. 30, 2002.

(51) Int. Cl. C12Q 1/68; G06F 19/00; G01N 33/48; G01N 33/50

(52) U.S. Cl. 435/6; 702/20

(57) ABSTRACT

The present invention is directed to the identification of genostatic factors, methods of determining the association of a plurality of genes with polygenic disorders, and method of assessing the sensitivity and specificity of the risk of polygenic disorders. In particular, the present invention discovers that the association between a polygenic disorder phenotype and polygenes may be masked. Incorporating genostatic factor, such as maternal age, birth disorder, the androgen receptor gene, gender, and age, into the statistical analysis of the association between phenotypes and genotypes reveals statistically significant relationship between the two. Accordingly, the present invention provides novel methods in determining the association of a plurality of genes with a polygenic disease and the likelihood of having the polygenic disease.

### CROSS-REFERENCE TO RELATED APPLICATIONS

This application claims priority to U.S. Provisional Patent Application Serial No. 60/407,341, filed Aug. 30, 2002, the disclosure of which is incorporated by reference herein in its entirety, including drawings.

### FIELD OF INVENTION

The present invention relates to the risk assessment of polygenic disorders. In particular, the present invention is directed to the identification of genostatic factors, methods of determining the association of a plurality of genes with polygenic disorders, and method of assessing the sensitivity and specificity of the risk of polygenic disorders.

### BACKGROUND

Genetic factors are involved in almost all disorders. In single gene disorders each mutation accounts for 100% of the variance from the norm. However, single gene disorders account for less than 2% of all disease morbidity. In contrast, polygenic disorders account for 98% of the genetic morbidity in humans. Virtually all humans are at risk during their lifetime for one or more polygenic disorders. Not surprisingly, billions of dollars have been spent by government and private industry in an attempt to decipher the genetics of a wide range of polygenic disorders; yet very little success has been achieved so far.

Polygenic disorders are due to the additive effect of multiple polygenes, which refers to the genes that contribute to a given polygenic disorder,¹⁷ each with a small effect interacting with the environment. Studies have shown that, while linkage and sibling pair analyses have succeeded in identifying the loci in single gene disorders, they generally lack the power to identify the polygenes involved in polygenic disorders. Association studies using either the family based Transmission Distortion Test (TDT) or population based case control studies do have the requisite power⁶⁰. However, because the effect of each gene in a polygene disorder is often small and there is a considerable genetic heterogeneity, there is considerable variability from study to study. Further, it has become clear that variability is just as great with family based as with population based studies, suggesting that population stratification may not be the culprit. This variation and history of poor replication has cast a pall over studies of polygenic disorders with many investigators believing the field is a hopeless area of study. Since the TDT technique requires samples on both parents and an affected child and is most powerful where there is one heterozygous and one homozygous parent, independent of its ability to negate possible population stratification, it is less powerful than case control association studies (Morton, N. E. & Collins, A., Tests and estimates of allelic association in complex inheritance, Proc Natl Acad Sci U S A 95: 11389-93 (1998)). In addition, because of the need for a heterozygous parent for each gene, if the additive effect of multiple genes is to be examined, the TDT technique is dramatically weakened compared to case controls studies.

Case control studies are also especially valuable in cases where certain individuals are affected, such as Alzheimer's disease or cancer, or behavioral disorders, where as a result of family conflicts, locating both parents may be difficult. Thus, for many, and possibly all polygenic disorders, especially if the additive effect of multiple genes is to be examined, case control studies are the most powerful approach to the identification of the causative genes. However, concerns about the potential for population stratification remain. To counter this, several authors have proposed genotyping a number of non-candidate genes to identify and correct for possible population stratification.^{32, 33, 58, & 76}

One issue is the effect size or the percent of the variance that can be attributed to each polygene. One method of measuring the effect size of a biallelic variation of any gene is to compute the Pearson correlation, r, between a given phenotype and the genotypes (11, 12, and 22) scored such that the genotypes showing the least effect are scored 0, those with the greatest effect are scored 2, and the remaining genotype is scored 0, 1 or 2. The fraction of the variance is r². In previous studies of several hundred genotype-phenotype associations, even when significant, the percent of the variance attributable to each gene generally ranged from 0.5 to 2.0 percent (r²=0.005 to 0.02) and averaged less than 1.5 percent,^{22, 23} even in disorders that are 50 to 70 percent genetic. This implies the potential involvement of many polygenes. Another method of measuring effect size is the use of odds ratios. For polygenic disorders each significant gene usually shows an odds ratio of 1.5 to 2.5.

Another major characteristic of polygenic disorders is great genetic heterogeneity. Thus, the same phenotype may be caused by many different combinations of polygenes. It has been proposed that the interaction of these two characteristics, a small percent of the variance attributable to each gene and great genetic heterogeneity, is the true cause of the variation from study to study and is the expected outcome for polygenic disorders (Comings D E (2002), The Real Problem with Association Studies, Am. J. Med. Genet. (Neuropsychiatric Genetics) (in press)). Thus, instead of focusing on single genes and imperfect replication from study to study, this phenomenon should be recognized as an integral and expected outcome for polygenic disorders and objective should instead focus on developing methods that take the unique characteristics of single genes into consideration. One of the most challenging aspects of polygenic disorders is that they are due to the additive effect of multiple genes, each of which has only a small effect, and there is considerable heterogeneity such that the involved genes may differ from one study group to another. Different sets of genes may produce the same phenotype. As a result, when genes are examined one-at-a-time the results are poorly replicable.

In considering the two characteristics of polygenic disorders, small effect size and genetic heterogeneity, into polygenic risk assessment, one potential approach to examining polygenic disorders is to examine the total variance of functionally related genes. In this approach, the additive effect of r² values of each gene of a number of functionally related genes²¹⁻²⁴ are examined and compared with relative effect of different groups of genes for the disorder in question. A number of different but functionally related genes can each contribute to the same phenotype. For example, several different dopamine genes, DRD1 (Comings et al., Studies of the potential role of the dopamine D1 receptor gene in addictive behaviors, Molecular Psychiatry 2:44-56 (1997)), DRD2 (Comings et al., The dopamine D2 receptor locus as a modifying gene in neuropsychiatric disorders J.Am.Med.Assn. 266:1793-1800 (1991)); DRD3 (Comings et al., Association of the dopamine DRD3 receptor gene with cocaine dependence, Molecular Psychiatry 4:484-487 (1999)); DRD4 (Lahoste et al., Dopamine D4 receptor gene polymorphism is associated with attention deficit hyperactivity disorder, Molecular Psychiatry 1:121-124 (1996); Rowe et al., Dopamine DRD4 receptor polymorphism and attention deficit hyperactivity disorder, Molecular Psychiatry 3:419-426 (1998); Faraone et al., Dopamine D4 gene 7-repeat alleles and attention deficit hyperactivity disorder, American Journal of Psychiatry 156:768-770 (1999)); DRD5 (Daly et al., Mapping susceptibility loci in attention deficit hyperactivity disorder: Preferential transmission of parental alleles at DAT1, DBH and DRD5 to affected children Molecular Psychiatry 4:192-196 (1999)), SLC6A3; DAT1 (Ebstein et al., Excess dopamine D4 receptor exon III (DRD4) seven repeat allele in opioid dependent subjects, Am.J.Hum.Genet. 59:A92 (1996); Comings et al., Polygenic inheritance of Tourette syndrome, stuttering, ADHD, conduct and oppositional defiant disorder: The Additive and Subtractive Effect of the three dopaminergic genes—DRD2, DBH and DAT1, Am. J.Med. Gen. (Neuropsych. Genet.) 67:264-288 (1996); Gill et al., Confirmation of association between attention deficit disorder and a dopamine transporter polymorphism, Molecular Psychiatry 2:311-313 (1997); Waldman et al., Association and linkage of the dopamine transporter gene and attention-deficit hyperactivity disorder in children: Heterogeneity owing to diagnostic subtype and severity, Am.J.Hum.Genet. 63:1767-1776 (1998)); DOC (Ernst et al., DOPA decarboxylase activity in attention deficit hyperactivity disorder adults. A [fluorine-18] fluorodopa position emission tomographic study, J.Neuroscience 18:5901-5907 (1998)); SLC18A1, and SLC18A2 (Russlee et al., Differences between electrically-ritalin-and D-amphetamine-stimulated release of [³H]dopamine from brain slices suggest impaired vesicular storage of dopamine in an animal model of Attention-Deficit Hyperactivity Disorder, Behav. Brain Res. 94:163-171 (1998)) have all been implicated in attention deficit hyperactivity disorder or its comorbid conditions, such as substance abuse. However, replication from study to study has been far from perfect. This suggests that a more reasonable approach is to examine the variance of each dopamine gene and compare the sum of the variance for all dopamine genes in subjects with the phenotype versus controls. The potential addictive or suppressive interaction between the dopamine genes could also be included in the total variance. To further characterize a given disorder, the relative total variance of genes belonging to different functional groups of genes, such as dopamine, serotonin, norepinephrine, GABA, opioid, and cholinergic, can be examined for different phenotypes. This approach has been used in the examination of the role of multiple genes in ADHD, oppositional defiant disorder (ODD), conduct disorder (CD), pathological gambling, and personality traits (Comings D E, Gade-Andavolu R, Gonzalez N, Wu S, Muhleman D, Blake H et al. Comparison of the role of dopamine, serotonin, and noradrenaline genes in ADHD, ODD and conduct disorder: multivariate regression analysis of 20 genes.^{23, 24} (See also, Comings D E and MacMurray J, Maternal age as a confounding variable in association studies, Am. J. Medical Genetics Neuropsychiatric Genetics 2001;105:564.).

Another potential approach to determine contribution to polygenic disorders is to determine multigene additive scores by Receiver Operator Characteristic (ROC) plots. This approach involves the examination of risk scores based on the additive effect of different candidate genes using ROC plots to determine the specificity and sensitivity of the resulting risk score.²⁹ Analysis by a total variance approach may require the examination of hundreds of genes. While this is increasingly practical with high throughput genotyping techniques, some disorders may be more efficiently studied by examining the additive and additive/suppressive effect of a small number of functionally different candidate genes. It is often the case that a number of genes may have already been implicated in a given disorder and while some show good or excellent replication across multiple studies, the results for others may show a mixture of replication and non-replication.

While these approaches have been powerful additions to risk assessments for polygenic disorders, only few polygenes have been identified. It is possible that the association between polygenes and polygenic disorder phenotype may be masked by some hidden variables or factors. Therefore, there is a need to identify additional variables that are associated with polygenic disorders and incorporating these variables in assessing the risk of polygenic disorders.

### SUMMARY OF INVENTION

One aspect of the invention is directed to the identification of genostatic factors in polygenic disorders.

Another aspect of the invention is directed to a method of identifying a genostatic factor in a polygenic disorder which comprises identifying the genotypes of a polygene, performing a statistical analysis for interaction between a variable and the genotype, and determining whether the variable is a genostatic factor if the analysis is statistically significant.

Another aspect of the invention is directed to a method of determining the association between a gene and a polygenic disorder which comprises the steps of identifying the genotypes of the gene, performing a statistical analysis for the interaction between the genotypes and a genostatic factor, and choosing the gene to be a polygene if the analysis is of statistical significance.

Another aspect of the invention is directed to a method of assessing the risk of polygenic disorders in the presence of a plurality of polygenes which comprises the steps of a) performing a statistical analysis for genostatic effect of a plurality of genostatic factors on each polygene; b) choosing the polygene if the genostatic effect is statistical significant; c) scoring the genotypes of the polygene of (b) in the presence of the genostatic factors, d) calculating the total variance of a plurality of polygenes of (c); e) retaining polygenes of (d) with statistical effect; f) computing a composite risk score of all the polygenes of (e); and h) evaluating the sensitivity and specificity of the risk of the polygenic disorders.

Another aspect of the invention is directed to a computer product comprising a computer program, which once executed by a computer processor performs methods as described in the present invention.

Other aspects of the invention are described in the specification, drawings, examples and claims herein.

### BRIEF DESCRIPTION OF THE DRAWINGS

FIG. 1 shows the additive and epistatic effects of two genes A and B.

FIG. 2 shows a scheme of genostasis.

FIG. 3 shows two-way interaction between variants of the leptin (LEP) gene and maternal age with age of onset of menarche. From Comings et al.²⁵

FIG. 4 shows two-way interaction of the DRD1 gene and maternal age with obsessive compulsive disorder (OCD) in the Tourette Syndrome (TS) database.

FIG. 5 shows two-way interaction of the DRD1 gene and maternal age with general anxiety disorder in the TS database.

FIG. 6 shows two-way interaction of OCD by maternal age with blood tryptophan levels in the TS database.

FIG. 7 shows two-way interaction of the DRD1 gene and birth order and OCD in the maternal age≧26 years group in the TS database.

FIG. 8 shows two-way interaction of the DRD1 gene and birth order with OCD in the maternal age≦5 years group in the TS database.

FIG. 9 shows three-way interaction of the DRD1 gene, maternal age and birth order with Attention Deficiency Hyperactivity Disorder (ADHD) in the Minnesota Twin & Family Study (MTFS) database.

FIG. 10 shows two-way interaction of the DRD4 and Androgen Receptor (AR) genes with Oppositional Defiant Disorder (ODD) in the TS database.

FIG. 11 shows two-way interaction of the DRD4 and AR genes with novelty seeking based on the Temperament Character Inventory (TCI) in the College student and Substance Use Disorder (SUD) databases.

FIG. 12 shows two-way interaction of the DRD2 and AR genes with novelty seeking in the college student and SUD databases.

FIG. 13 shows two-way interaction of the DRD2 and AR genes and depression in women in the obesity

FIG. 14 shows two-way interaction of the HTR2C and AR genes and paranoid personality in the SUD database.

FIG. 15 shows two-way interaction of the AR and COMT genes with Tics in the TS database.

FIG. 16 shows interaction of the COMT gene and birth order with tics in TS probands of maternal age≧26 years and AR≦16 alleles in the TS database.

FIG. 17 shows two-way interaction of the ADRB2 and AR genes with NIDDM in the obesity database.

FIG. 18 shows two-way interaction of the AR and 11B-HSB1 genes with cholesterol in the obesity database.

FIG. 19 shows Receiver Operator Characteristic (ROC) plot for the additive effect of four candidate genes associated with sporadic breast cancer.

### DETAILED DESCRIPTION OF THE INVENTION

Genostatic factors can modify and even reverse the association of given genotypes with a given polygenic disorder. In addition, genostatic factors may mask the association of genotypes with polygenic disorders. Such masking may have given rise to the contradictory outcomes of many genetic studies to date. Therefore, inclusion of genostatic factors in genetic studies will allow a more thorough examination of datasets.

Accordingly, one aspect of the present invention relates to the identification of such genostatic effects and methods that can be used to reveal genostatic effects present within genotyping studies, thereby unmasking genotype variation that may be overlooked and/or not be detectable using techniques currently known in the art (e.g., case-matched controls, twin studies and sibling pair studies).

In a preferred embodiment of the invention, a further layer of complexity in genetic analysis is added due to epistatic effects, including epistatic effects that are reliant on a genostatic factor being present. Analysis of both epistatic and genostatic effects is described according to methods herein and examples are given where significant associations with genotypes can be extracted from datasets when epistatic an/or genostatic factors are considered.

Epistasis used herein refers to gene-gene interactions. For example, genes A and B may each account for only a small and non-significant percent of the variance of a given trait (ex: r²=0.005, p=non-significant (N.S.)), but the interaction of the two genes may account for a much higher and significant percent of the variance (ex: r²=0.05, p≦0.001). The typically described type of epistasis involves a greater than additive (or subtractive) effect of two genes.

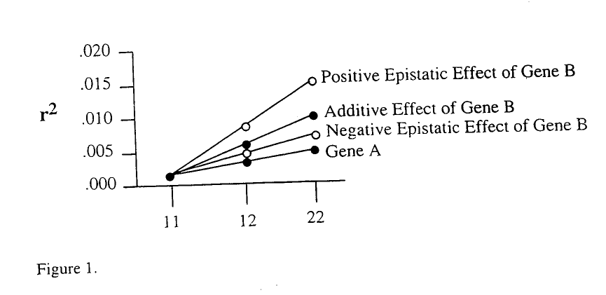

An example of an epistatic effect is shown in FIG. 1. Here, for 22 genotypes, genes A and B each produce and r² of 0.005. The sum of the r² of for both genes is 0.01. However, if there is an epistatic effect, the r² of the two genes together would be significantly greater than 0.01. In this case the epistatic effect is 0.015. In terms of odds ratios, the odds ratio for gene A might be 1.5, of gene B 1.5 but in the individuals carrying the 22 genotype of both genes, the odds ratio might be 4.5.

As a further example, in a study of susceptibility to osteoporotic fracture, a specific haplotype of the COL1A1 gene was associated with a 1.8 odds ratio in heterozygotes and a 2.6 odds ratio in homozygotes.⁶⁸ A polymorphism at the Vitamin D receptor gene was not significantly associated with and increased odds ratio for fracture in those not carrying the COL1A1 risk haplotypes. However, in presence of the Vitamin D variant and heterozygosity for the COL1A1 variant the odds ratio was 2.1 and in the presence of homozygosity for the COL1A1 variant the odds ratio was 4.4. Thus, the epistatic effect of the two genes was greater than the simple additive effect of the genes separately.

Genostasis used herein refers to a situation in which the presence of condition B of a factor or a variable reverses the effect of the genotypes of gene A on a given phenotype, while the presence of condition A enhances the effect. This is in contrast to epistasis where the normal condition (allele) the second gene has no effect, while the variant allele has a positive (or negative) effect on the genotypes of the first gene. The factor or the variable resulting a genostatic effect is viewed as a genostatic factor. For genostasis, the genostatic factor can be a gene or a non-gene demographic or environmental variable. If not identified these could be referred as ‘hidden’ modifying variables.

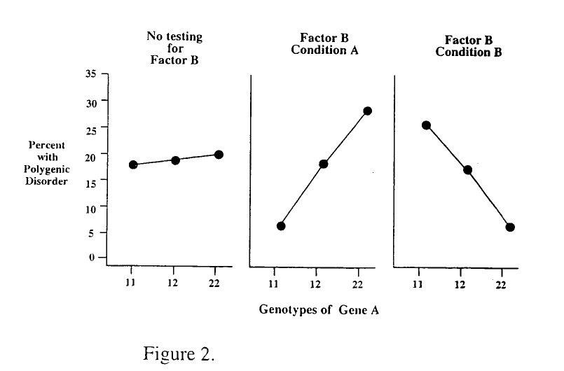

The effect in genostasis or genostatic effect of a genostatic factor on gene A is illustrated in FIG. 2. The left panel shows that when the effect of the hidden or genostatic factor (Factor B) is ignored, gene A appears to have either no effect on the phenotype or such a mild effect that the results are variable from study to study. However, in the presence of condition A of the genostatic factor (Factor B), there is a progressive increase in the frequency of the polygenic disorder from genotype 11 to 12 to 22 (2-allele codominant). By contrast, in the presence condition B of Factor B, the effect is reversed such that now the highest frequency of the disorder is associated with genotype 11 with progressive decreases for genotypes 12 and 22 (1-allele codominant). The term genostasis was coined to represent the genotype (geno-) reversal or neutralization (-stasis) of genostatic factor (Factor B) on Gene A.

Accordingly, in addition to a small effect size for each gene and genetic heterogeneity, genostatic factors constitute a major characteristic of polygenic disorders that accounts for much of the variability from study to study. In a preferred embodiment of the invention, a genostatic factor is maternal age, birth order, a genostatic gene, gender, or age. In a more preferred embodiment of the invention, a genostatic factor is maternal age (age of the mother at the time of the birth of a proband), birth order, or a genostatic gene. It is preferred that a genostatic gene is a genetic variant at the androgen receptor gene.

Since factors such as maternal age, gender and age can be shown to be important to genostasis, it is contemplated that other biological factors such as the generation, maintenance and catabolism of hormones, body weight, bone mass and other factors, especially systemic factors, may also be involved in genostatic effects. Indeed, body mass index, bone density, ratio of “fast-twitch”/slow-twitch” muscle fibers, resting heart rate, blood pressure are examples of biological features in the mother, or father, that might, using the statistical analysis described herein, relate directly or indirectly to genostasis. Likewise, exogenous factors such as stress, medications, drugs such as nicotene or marijuana may also have direct or indirect genostatic effects.

The identification of genostatic factors allow the development of methods to compute statistical relationship between a polygene, which is a gene contributes to a polygenic disorder, and a genostatic factor. When genostatic factors are included in risk assessment of polygenic disorders, genostasis increases the power of identifying the genes involved in polygenic disorders. The genostatic effect further allows the development of methods of assessing the risk of polygenic disorders of a given individual, which includes the steps of, for example, scoring each genotype to accommodate genostatic effects, computing composite risk scores (CRS) of all polygenes, and evaluating the sensitivity and specificity of the risk.

Developing a Database for Polygenic Disorders and Relevant Polygenes.

As known in the art, in assessing the risk of polygenic disorders, a database on polygenic disorders or diseases to be studied needs to be developed first. Blood, buccal smear or other samples need to be collected from cases and controls to allow isolation of DNA. Other data will include age and date of birth of the proband, age and date of birth of the mother of the proband (a proband refers to a person who is the initial member of a family to come under study for a polygenic disorder), age and date of birth of each sibling. This allows the determination of the maternal age and birth order of the proband. Obtaining the ages of birth is important in case the individuals involved have died. Ensuring that each subject in the database is of the same racial/ethnic background will help to minimize the risk of stratification effects. It is best if the number of subjects is great enough to provide adequate power for both an initial and a replication set. Empirically, at least 100 controls and 100 subjects in each set is desirable. This is a 10 to 30 fold lower number of cases than required for sibling pair analysis.^{59, 60} Larger numbers for two replication sets are preferable if a large number of candidate genes are to be tested.

Meanwhile, candidate genes suspectible of being associated with a polygenic disorder need to be identified or selected. The vast amount of biological information concerning a wide range of disorders, and the results of the human genome project, allow the identification of a large number of candidate genes for any given polygenic disorder. For example, important candidates for any behavioral or psychiatric disorder would at a minimum include the genes for each of the neurotransmitters, neuropeptides,²² neurosteroids,³⁹ hormones,²⁴ G-proteins, and secondary messengers. Autoimmune disorders would include at a minimum the cytokine and chemokine genes. While the candidate gene approach may miss some of the involved genes, it is likely that the methods of the present invention would account for a sufficiently high r² value as to be of great predictive and potentially therapeutic value. It is preferred that the inclusion of 2 to 3 polymorphisms at 10 or more non-candidate genes will allow the use of a range of techniques to rule out or correct for population stratification.^{32, 33, 58, 76}

Determining and Genotyping the Polymorphism or Alleles at Each of Candidate Genes.

If well validated polymorphisms for the genes in question are not available from the literature, excellent candidates can now be obtained from the SNP consortium (Locus Link). In the past there has been great concern that if a polymorphism was not in a critical parts of the gene such as promoters, splice sites, or reading frames, the polymorphisms would be of no use. It is now clear that the genome is divided into short segments consisting of a small number of haplotypes in which all the SNPs and other polymorphisms are in strong linkage disequilibrium, separated by regions of high recombination.^{31, 49} This indicates that only a few polymorphisms, with high frequency alleles, are needed to evaluate each gene.

Polymorphism of genes can be determined and genotyped by methods well known in the art. The determination can be carried out either as a DNA analysis according to well known methods, which include indirect DNA sequencing of the normal and mutated genes at said genetic loci, allele specific amplification using the polymerase chain reaction (PCR) enabling detection of either normal or mutated sequences at said genetic loci, or by indirect detection of the normal or mutated alleles at said genetic loci by various molecular biology methods including, e.g., PCR-single stranded conformation polymorphism (SSCP)-method or denaturing gradient gel electrophoresis (DGGE). Determination of the normal or mutated alleles at said genetic can also be done by single restriction fragment length polymorphism (RFLP) method.

Nucleic acid analysis via microchip technology is also applicable to the present invention. In this technique, literally thousands of distinct oligonucleotide probes can be applied in an array on a silicon chip. A nucleic acid to be analyzed is fluorescently labeled and hybridized to the probes on the chip. It is also possible to study nucleic acid-protein interactions using these nucleic acid microchips. Using this technique one can determine the presence of mutations, sequence the nucleic acid being analyzed, or measure expression levels of a gene of interest. The method is one of parallel processing of many, even thousands, of probes at once and can tremendously increase the rate of analysis.

Polynucleotide polymorphisms associated with particular alleles from candidate genes for a polygenic disorder can further be detected by hybridization with a polynucleotide probe which forms a stable hybrid with that of the target sequence, under highly stringent to moderately stringent hybridization and wash conditions. If it is expected that the probes will be perfectly complementary to the target sequence, high stringency conditions will be used. As well known by those of ordinary skill in the art, hybridization stringency may be lessened if some mismatching is expected, for example, if variants are expected with the result that the probe will not be completely complementary. Reaction conditions are chosen which rule out nonspecific/adventitious bindings, that is, which minimize noise.

Nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of 30° C., typically in excess of 37° C., and preferably in excess of 45° C. Stringent salt conditions will ordinarily be less than 1000 mM, typically less than 500 mM, and preferably less than 200 mM. However, the combination of parameters is much more important than the measure of any single parameter. The stringency conditions are dependent on the length of the nucleic acid and the base composition of the nucleic acid, and can be determined by techniques well known by persons of ordinary skill in the art.

The determination can also be carried out at the level of RNA by analyzing RNA expressed at tissue level using various methods. Allele specific probes can be designed for hybridization. Hybridization can be done e.g. using Northern blot, RNase protection assay or in situ hybridization methods. RNA derived from the normal or mutated alleles at said genetic loci can also be analyzed by converting tissue RNA first to cDNA and thereafter amplifying cDNA by an allele specific PCR-method and carrying out the analysis as for genomic DNA as mentioned above.

Alteration of mRNA transcription can be detected by any techniques known to persons of ordinary skill in the art, such as, by way of example, Northern blot analysis, PCR amplification and RNase protection. Diminished mRNA transcription can indicate an alteration of the wild-type gene.

Polymorphisms in a gene can also in some instances be detected by screening for alteration of the protein encoded by the gene. For example, monoclonal antibodies immunoreactive with an allele can be used to screen a tissue. Lack of cognate antigen would indicate absence of an allele. Antibodies specific for products of an allele also could be used to detect the product of the allele. Such immunological assays can be done in any convenient format known in the art. These include Western blots, enzyme linked immunosorbent assays (ELISA), radioimmunoassays (RIA), immunoradiometric assays (IRMA) and immunoenzymatic assays (IEMA). Any means for detecting an altered protein can be used to detect polymorphisms of gene. Functional assays, such as protein binding determinations, also can be used. In addition, assays which detect biochemical function can be used.

After the polymorphism or alleles of each candidate polygene is determined, statistical analyses for association of the genes and polymorphisms for the phenotype in question can be performed. This involves determining the frequency of the genotypes in cases with the disorder and unrelated, racially matched controls, or examination of the frequency of comorbid disorders within a group of individuals with a specific disorder by genotype, as described above.

Testing Genostatic Effects of a Genostatic Factor on the Genotypes of a Plurality of Polygenes.

In a preferred embodiment, the test is performed by Hierarchical Analysis of Variance (ANOVA). Hierarchical ANOVA allows the examination of the genostatic effect of genotype with each allele as a variable in the presence of a genostatic factor as an independent variable.²⁵ For example, the test of genostatic effects of maternal age can be easily performed by hierarchical ANOVA using the genotypes of the gene and maternal age groupings as the independent variables and testing for a gene by maternal age interaction effect. A significant genostatic effect is indicated when the interaction term is significant while the gene or maternal age groups alone is not significant or shows minimal significance.

The test for genostatic effects of birth order can be performed by hierarchical ANOVA using the genotypes of the gene and birth order groupings as the independent variables and testing for a gene by birth order interaction effects. If there was a significant gene by maternal age effect, the two maternal age groups should be tested independently for a gene by birth order effect.

The test for genostatic effects of a genostatic gene can be performed by hierarchical ANOVA using the genotypes of the gene A and gene B the independent variables. As described above, other genostatic genes can also produce genostatic effects. If the AR gene is chosen to examine for a potential gene A by the AR gene's genostatic effect, all subjects need to be genotyped at the AR gene. The same is true if any other genostatic gene is to be examined for a genostatic effect.

By the same token, the genostatic effect of gender as a genostatic factor can also be tested. In many cases the associations identified are gender specific and are significant in males but not females, or visa versa. In the case when age is used as a genostatic factor, the associations identified are age specific and are significant in young but not older probands, or pre-menopausal but not post-menopausal probands, or visa versa.

It is contemplated that genostatic effect can also be analyzed by other statistical methods including multivariate regression analysis, multivariate logistic regression analysis, three way chi square analysis and others.

When the genostatic effect of a genostatic factor on genotype, or the interaction between genotype and phenotype in the presence of the genostatic factor, is deemed as statistically significant, the polygene with the genotypes is chosen for further scoring. It is preferred that a genostatic effect is statistically significant when a p value is no more than 0.1 (P≦0.1). It is more preferred that a genostatic effect is statistically significant when a p value is no more than 0.05(P≦0.05). It is even more preferred that a genostatic effect is statistically significant when a p value is no more than 0.01 (P≦0.01). Varying levels of alpha (0.05, 0.01, 0.005 or others) can be chosen to decrease the risk of type I errors. Those genes that meet the chosen criteria are then used for the further analyses described below.

Scoring Each Gene/Polymorphism to Accommodate the Effects of Epistatic or Genostatic Factors.

One form of accommodation or incorporation of the effects of epistatic factors or genostatic factors of genostatic genes into an analysis is to perform simple stratification of patient genotypes and phenotypes into groups representing the presence or absence of the genostatic or epistatic factor, or by the presence or absence of the genostatic or epistatic factor relative to a cut-off or threshold. While a stratification analysis is used here to code for epistatic or genostatic factors, analyses could be performed that accommodated genostatic or epistatic factors that had a dynamic influence. Dynamic influence refers a factor whose contribution to the emergence of a phenotype from the given genotype is not fixed or may not be usefully stratified into a small number of groups. Examples of factor with dynamic influence could include, but would not be limited to, a factor that had an effect on the emergence of a phenotype that linearly increased with age, or a factor whose influence exponentially declined with, for example, each successive birth. One mode of this invention would be an analysis that could extract such dynamic factors for use in the method. One approach to achieving such an extraction would be by coding factors into multiple groups using small or point-sized intervals. Indeed, for the elucidation and incorporation of dynamic genostatic and epistatic factors in the analysis, specific calculations may need to be applied to the dataset (“data mining”) to best extract the relationship of these dynamic factors with the genotype and phenotype under examination. Such data mining techniques are well known in the art would include univariate and multivariate regression analysis, neural networks, transforming datasets from spatial domain into frequency domains (e.g using Fourier transform operations), cluster analysis and other pattern recognition techniques. Such techniques are commonly used in diverse fields such as statistics, physics, astronomy, image analysis, and signal processing but have common underpinnings that can be used to extract meaningful data from complex datasets and are of increasing use in the analysis of biological information in the field of bio-informatics.

In the examples given herein, coding based stratification is used to accommodate the effects of genostatic factors. An example of the SPSS syntax file used to code the DRD1 gene and to include the maternal age and birth order effects is shown as follows where DRD1 1=11, DRD1 2=12 and DRD1 3=22 genotypes, “moage2526=1” is maternal age≦25 years, “moage2526=2” is maternal age≧26 years, and “gMBOC_D1” represent the gene scores (gs or g) that include Maternal age effects (M), birth order effects (O) for the OCD phenotype (OC) at the DRD1 gene (_D1).

***coding for genotype effect with maternal age and birth order

if (moag2526 eq 1) and (DRD1 eq 1) gMBOC_D1=0.

if (moag2526 eq 1) and (DRD1 eq 2) gMBOC_D1=1.

if (moag2526 eq 1) and (DRD1 eq 3) gMBOC_D1=2.

if (moag2526 eq 2) and (birth_or eq 1) and (DRD1 eq 1) gMBOC_D1=2.

if (moag2526 eq 2) and (birth_or eq 1) and (DRD1 eq 2) gMBOC_D1=0.

if (moag2526 eq 2) and (birth_or eq 1) and (DRD1 eq 3) gMBOC_D1=0.

if (moag2526 eq 2) and (birth_or eq 2) and (DRD1 eq 1) gMBOC_D1=2.

if (moag2526 eq 2) and (birth_or eq 2) and (DRD1 eq 2) gMBOC_D1=2.

if (moag2526 eq 2) and (birth_or eq 2) and (DRD1 eq 3) gMBOC_D1=0.

if (moag2526 eq 2) and (birth_or eq 3) and (DRD1 eq 1) gMBOC_D1=0.

if (moag2526 eq 2) and (birth_or eq 3) and (DRD1 eq 2) gMBOC_D1=2.

if (moag2526 eq 2) and (birth_or eq 3) and (DRD1 eq 3) gMBOC_D1=1

A similar code can be used to incorporate the effects of other modifying factors such as gender, age, race, and others. Other statistical programs that allow syntax files, such as SAS, can also be used.

Computing the Total (Variance) r² for All of the Included Polygenes.

This is done by using the phenotype score (a continuous variable or control=0 and subject=1) as the dependent variable and the gene scores for each of the selected genes as the independent variables in a multivariate regression analysis if the dependent variable is continuous or a multivariate logistic regression analysis if the dependent variable is dichotomous. This allows the estimation of the total r² and p value for the whole set of genes. If the r² is large (0.2 or greater) or the p value is small (0.1 or less; 0.05 or less), it is likely that a successful predictive test can result and the procedure continues with the following steps. This step can also be used to farther identify those genes which retain a significant effect (individual p of ≦0.05 or ≦0.01) on the phenotype in the presence of all the other included genes.

Computing a Composite Risk Core for All of the Included Polygenes.

This process adds together the genes scores for each individual for all of the included genes to arrive a total score—a composite risk score or CRS. This is a simple addition process. An example of the SPSS code for this is as follows (gsGeneA eq 1 means that the gene score for Gene A equals to 1):

compute CRS=0.

if (gsGeneA eq 1) CRS=CRS+1.

if (gsGeneA eq 2) CRS=CRS+2.

if (gsGeneB eq 1) CRS=CRS+1.

if (gsGeneB eq 2) CRS=CRS+2.

if (gsGeneC eq 1) CRS=CRS+1.

if (gsGeneC eq 2) CRS=CRS+2.

if (gsGeneD eq 1) CRS=CRS+1.

if (gsGeneD eq 2) CRS=CRS+2.

if (gsGeneE eq 1) CRS=CRS+1.

if (gsGeneE eq 2) CRS=CRS+2.

Etc.

Evaluating the Sensitivity and Specificity of the CRS.

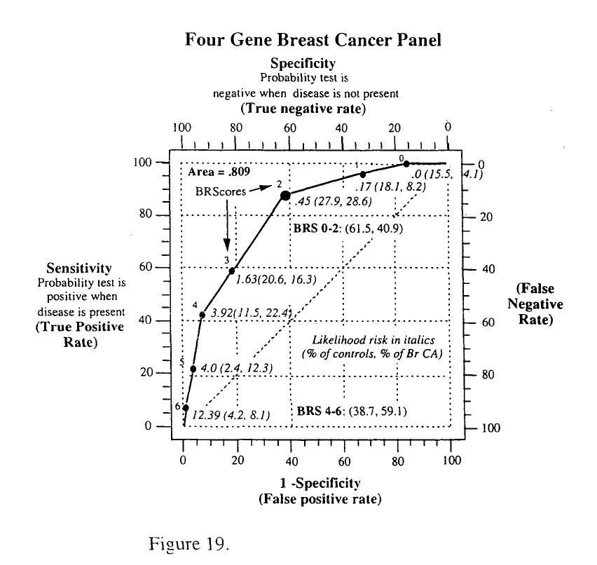

Receiver Operator Characteristic (ROC) plots are used to evaluate the sensitivity and specificity of the CRS. Receiver Operator Characteristic (ROC) plots provide a pure index of the accuracy of a given test by demonstrating the limits of the tests ability to discriminate between alternative states of health or disease over the complete spectrum of operating conditions.^{56, 77} The ROC plots are also described in the U.S. patent application Ser. Nos. 10/401,132 and 10/319,855, which are incorporated by reference in their entirety. The ROC plot depicts the overlap between the two distributions by plotting the sensitivity versus specificity for the complete range of decision thresholds. Computer programs considerably enhance the ease of use of ROC curves.⁷⁷ These programs allow the determination of the positive and negative likelihood ratios for the presence of disease for each of the sensitivity-specificity pairs. The product of the two, termed here the likelihood risk, is useful since those who a have neutral risk have scores of approximately 1, those with a diminished risk have scores less than 1, and those with a higher risk have scores of greater than 1. The program also calculates the area under the curve, a measure of the effectiveness of the test.⁴⁵ The following (FIG. 19) is an example of a four gene risk assessment for breast cancer using the above procedure (except for the inclusion of maternal age and birth order).

FIG. 19 illustrates how the genotypes at several breast cancer risk genes can be combined into a ROC plot to produce a clinically useful guide for a given woman about her risk for breast cancer. The numbers above the line represent the different CRS scores. The area under the curve is 0.809. This relatively large value indicates that test is clinically useful. The numbers under the curve represent the relative risk figures. The lower the figure the lower the risk. In this case they ranged from 0.0 for a CRS of 0, to 12.39 for a CRS of 6. The same ROC curves can be plotted for any polygenic disorder with the CRS developed as described above.

To avoid the problem of circular reasoning, in which the data for the gene scores is derived from the same database used for the CRS scores and assessment by ROC curves, the same gene scores need to be used in a second replicate set to ensure reproducibility. If available, the use of a third test set is also desirable.

Significant efforts have been put in attempts to identify the genes involved in the common, complex, polygenic disorders. However, with only a few exceptions, methods available in the art have failed to identify the relevant genes. It is contemplated that genostatic factors may account for the failure since these factors may mask or module the emergence of the phenotype from a given genotype. The present invention teaches a powerful method for the identification of genes involved in complex polygenic disorders using genostatic factors. When these factors are taken into account for the scoring of candidate genes it is possible to develop composite risk scores that can be assessed in ROC plots. Until the methods in the present invention were demonstrated, approaches currently available in the art, which fail to take these genostatic factors into consideration, did not give rise useful outcomes in assessing the risk of polygenic disorders.

Computer Program and/or Product.

In a preferred embodiment, the methods described herein can be performed through the use of a computer system. Accordingly, another aspect of the present invention is directed to a computer software program which, once executed by a computer processor, performs methods as described herein. Yet another aspect of the present invention is directed to a computer program product comprising a computer software program which, once executed by a computer processor, performs the methods as described herein.

A computer system according to the present invention refers to a computer or a computer readable medium designed and configured to perform some or all of the methods as described herein. A computer used herein may be any of a variety of types of general-purpose computers such as a personal computer, network server, workstation, or other computer platform now or later developed. As commonly known in the art, a computer typically contains some or all the following components, for example, a processor, an operating system, a computer memory, an input device, and an output device. A computer may further contain other components such as a cache memory, a data backup unit, and many other devices. It will be understood by those skilled in the relevant art that there are many possible configurations of the components of a computer.

A processor used herein may include one or more microprocessor(s), field programmable logic arrays(s), or one or more application specific integrated circuit(s). Illustrative processors include, but are not limited to, Intel Corp's Pentium series processors, Sun Microsystems' SPARC processors, Motorola Corp.'s PowerPC processors, MIPS Technologies Inc.'s MIPs processors, Xilinx Inc.'s processors, and Vertex series of field programmable logic arrays, and other processors that are or will become available.

An operating system used herein comprises machine code that, once executed by a processor, coordinates and executes functions of other components in a computer and facilitates a processor to execute the functions of various computer programs that may be written in a variety of programming languages. In addition to managing data flow among other components in a computer, an operating system also provides scheduling, input-output control, file and data management, memory management, and communication control and related services, all in accordance with known techniques. Exemplary operating systems include, for example, a Windows operating system from the Microsoft Corporation, a Unix or Linux-type operating system available from many vendors, another or a future operating system, and some combination thereof.

A computer memory used herein may be any of a variety of known or future memory storage devices. Examples include any commonly available random access memory (RAM), magnetic medium such as a resident hard disk or tape, an optical medium such as a read and write compact disc, or other memory storage device. Memory storage device may be any of a variety of known or future devices, including a compact disk drive, a tape drive, a removable hard disk drive, or a diskette drive. Such types of memory storage device typically read from, and/or write to, a computer program storage medium such as, respectively, a compact disk, magnetic tape, removable hard disk, or floppy diskette. Any of these computer program storage media, or others now in use or that may later be developed, may be considered a computer program product. As will be appreciated, these computer program products typically store a computer software program and/or data. Computer software programs typically are stored in a system memory and/or a memory storage device.

An input device used herein may include any of a variety of known devices for accepting and processing information from a user, whether a human or a machine, whether local or remote. Such input devices include, for example, modem cards, network interface cards, sound cards, keyboards, or other types of controllers for any of a variety of known input function. An output device may include controllers for any of a variety of known devices for presenting information to a user, whether a human or a machine, whether local or remote. Such output devices include, for example, modem cards, network interface cards, sound cards, display devices (for example, monitors or printers), or other types of controllers for any of a variety of known output function. If a display device provides visual information, this information typically may be logically and/or physically organized as an array of picture elements, sometimes referred to as pixels.

As will be evident to those skilled in the relevant art, a computer software program of the present invention can be executed by being loaded into a system memory and/or a memory storage device through one of input devices. On the other hand, all or portions of the software program may also reside in a read-only memory or similar device of memory storage device, such devices not requiring that the software program first be loaded through input devices. It will be understood by those skilled in the relevant art that the software program or portions of it may be loaded by a processor in a known manner into a system memory or a cache memory or both, as advantageous for execution.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means without the exercise of inventive capacity and without departing from the scope of the invention.

### EXAMPLES

### Example 1

LEP×Maternal Age with Age of Menarche in the Obesity Database.

Our interest in the potential role of maternal age in human genetics was stimulated by the study of mice by Wang et al⁷³ entitled Maternal age and traits in offspring. They reported that the body, testes, and epididymis weight of 3 month old male offspring (F1 generation) was significantly higher for mothers of medium maternal age compared to offspring of mothers at lower or higher maternal age. They also observed that during pregnancy, serum estradiol was significantly higher in the mothers of medium maternal age compared to mothers of low or high maternal age. During pregnancy, serum testosterone was higher in the lower and medium maternal age mothers than in these with a high maternal age. These maternal age effects were also shown in the age of completed puberty of the F1 generation females. This age was significantly delayed in the F1 females of mothers of low and high maternal age compared to F1 females of medium maternal age mothers.

Of particular interest, these effects also persisted into the F2 generation. The birth weight of F2 pups of medium maternal age grandmothers was significantly greater than the F2 pups of low and high maternal age grandmothers. The authors cited evidence that hormones in utero may permanently ‘imprint’ the function of cells in the reproductive organs, the brain and many other tissues^{46, 72} and pointed out that with the exception of the role of advanced maternal age on aging oocytes, there were few studies in humans of maternal age effects.

The involvement of reproductive variables in the Wang et al report stimulated us to examine the potential role of the leptin gene (LEP) and maternal age on the onset of menarche in women. Studies in mice have indicated that leptin plays an important role in initiating puberty.²¹ These studies suggest that leptin is the signal that informs the brain that energy stores in the form of fat are sufficient to support the high energy demands of reproduction.¹ Conversely, they also suggest that in times of fasting, infertility induced by low leptin levels protects the female from the energy demands of pregnancy.^{13, 51} There is also much evidence for a major role for leptin in the initiation of puberty in humans.^{13, 41, 54, 74 5, 7, 9, 42, 51, 61} Missense mutations of the LEP gene⁶⁶ and the leptin receptor gene¹⁴ are associated with hypogonadism and obesity. The effect of increased leptin levels on the initiation of puberty appears to be secondary to the suppression of neuropeptide Y by leptin,⁵¹ thus releasing its inhibition of the pituitary-gonadotropin axis.

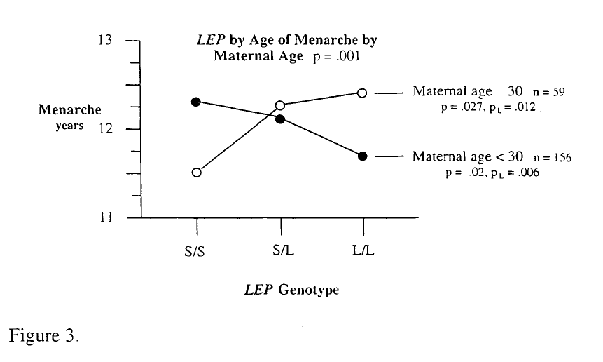

Several dinucleotide repeat polymorphisms in or near the human LEP gene have been identified.⁴⁴ An association between the D7S1875 polymorphism of the LEP gene and obesity in young females was reported in 1996.²⁷ The distribution of the alleles at the D7S1875 dinucleotide repeat showed two major peaks with the shorter alleles (S) ranging from 199 to 207 bp in length, and the longer alleles (L) ranging from 208 to 225 bp in length. Using the same polymorphism, Butler et al⁸ confirmed an association with BMI. For this study we used our obesity database designed to study the genetics of obesity.²⁷ There was no association of the LEP genotypes with the age of onset of menarche. However, when maternal age (age of the mother at the time of the birth of the proband) was included as a latent factor the results were quite different. FIG. 3 shows the results of this study of the association between the LEP gene based on S/S, S/L and L/L genotypes of the leptin gene and age of menarche.

When the cases were divided into maternal age of <30 versus ≧30 years, the associations were in the opposite direction. For maternal age <30 years, early age of onset of menarche were significantly associated with the L alleles, while for maternal age ≧30 it was significantly associated with the S alleles. Using a break point of maternal age of 25/26 years gave the same results. If maternal age was not taken into consideration, there was no apparent effect of the LEP gene on the age of onset of puberty.

### Example II

DRD1×Maternal Age with OCD in the Tourette Syndrome Database.

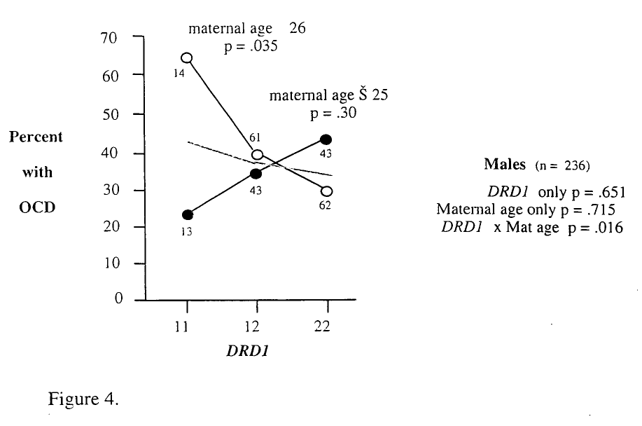

To address whether maternal age has an effect on the association of other genes with other phenotypes, we chose to first examine the role of maternal age as a confounding factor in the association of the DRD1 gene with obsessive compulsive disorder and other disorders. Knockout studies have implicated the DRD1 gene in obsessive compulsive disorders (OCD).³⁴ To test this we utilized our DNA database of Tourette syndrome subjects. This database consists of DNA, psychiatric assessments, and pedigrees on a large number of individuals with Tourette syndrome (TS), in whom OCD and other disorders are common comorbid conditions.^{9, 20, 40} The extensive pedigrees allow the determination of maternal age, birth order and related variables. FIG. 4 shows these results.

For probands with a maternal age of ≦25 years there was a 2 allele codominant relationship between the percent with comorbid OCD and the DRD1 gene. This was reversed in probands with maternal age ≧26. Now the association of the DRD1 gene with OCD was 1 allele codominant. The dotted line shows the non-significant association of the DRD1 gene when maternal age was not considered. Neither the DRD1 gene alone (p=0.651) nor maternal age alone (p=0.715) was significant, while the DRD1 gene by maternal age interaction was significant (p=0.016). In this case the genostasis effect on OCD was due to the non-gene variable of maternal age.

### Example III

DRD1×Maternal Age with General Anxiety Disorder in TS Database.

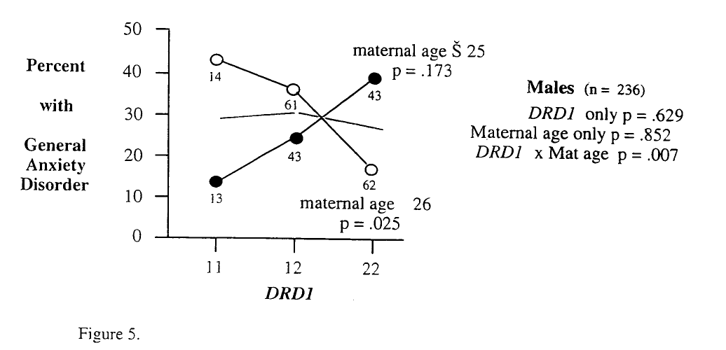

Since the knockout mice also suggested an association of the DRD1 gene with anxiety⁷¹ we also examined the presence or absence of general anxiety disorder (GAD) in the TS probands. These results are shown in FIG. 5.

The results for GAD were similar to those for OCD. For probands with a maternal age of ≦25 years there was a 2-allele codominant relationship between the percent with comorbid GAD and the DRD1 gene. This was reversed in probands with maternal age ≧26. Now the association of the DRD1 gene with GAD was 1 allele codominant. The dotted line shows the non-significant association of the DRD1 gene when maternal age was not considered. Neither the DRD1 gene alone (p=0.629) nor maternal age alone (p=0.852) was significant, while the DRD gene by maternal age interaction was significant (p=0.007). In this case the genostasis effect on GAD was due to the non-gene variable of maternal age.

### 94 Example IV

OCD×Maternal Age with Tryptophan in the TS Database.

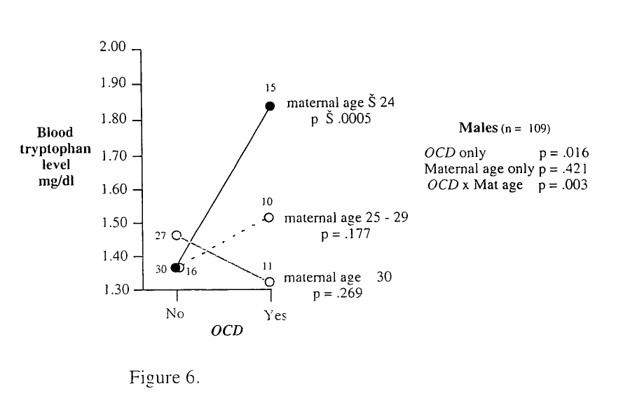

Defects in serotonin metabolism have long been implicated in OCD⁵⁷ and selective serotonin re-uptake inhibitors (SSRIs) are the drugs of choice in the treatment of OCD. Since tryptophan is the precursor of serotonin we examined the potential association between blood tryptophan levels and the presence or absence of OCD in TS probands. FIG. 6 shows the importance of maternal age in the relationship between a biochemical value (blood tryptophan) and OCD.

There was a strong association between blood tryptophan levels and the presence of OCD in probands with maternal age of ≦24. This was less marked in probands with maternal age 25-29, and reversed in probands with maternal age ≧30.

### Example V

DRD1×Maternal Age and Birth Order with OCD in the TS Database.

One potential mechanism to explain these findings is that there may be differences in methylation of genes secondary to variation in the hormonal milieu of the uterus for mothers of different maternal age. We reasoned that if this was true of maternal age it should also be true of birth order. While birth order and maternal age increase together, birth order could have an effect independent of its relationship to maternal age.

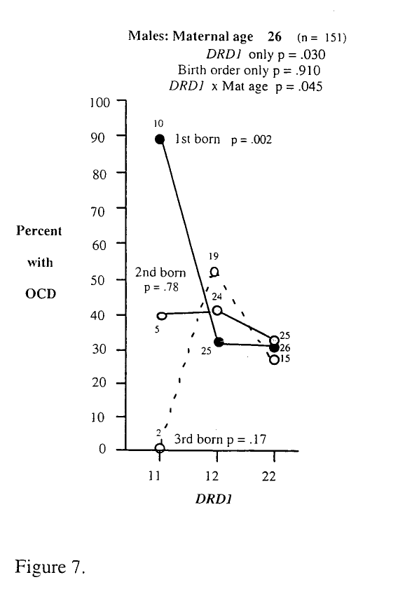

The effect of maternal age and birth order on the association of the DRD1 gene with OCD was examined in the TS database. FIG. 7 first shows the results for the maternal age ≧26 year group.

FIG. 7 showed there was a dramatic effect of birth order on the association of the DRD1 gene with OCD for probands with maternal age ≧26 years. In the first born probands OCD was most strongly associated with the 11 genotype of the DRD1 gene. By contrast, for the 3^{rd} born or later OCD was least associated with the 11 genotype. The 2^{nd} born probands showed an intermediate effect.

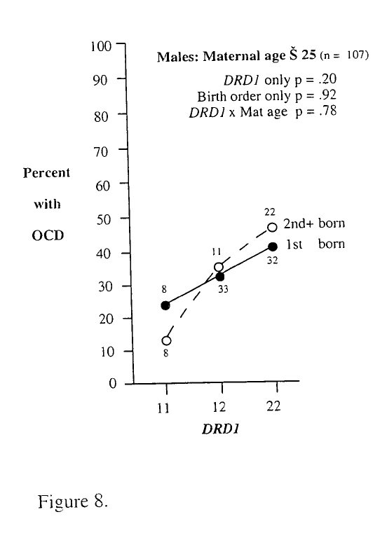

The effect of birth order in the probands with a maternal age of ≦25 years is shown in FIG. 8. Here, since the number of women who had their 3^{rd} child by 25 years of age was quite small, we only examined 1^{st} born versus 2^{nd}+ born.

In contrast to the ≦26 year group, in the ≦25 year maternal age group, birth order had no effect on the association of the DRD1 gene with OCD.

### Example VI

DRD1×Maternal Age×Birth Order with ADHD in the Minnesota Twins Database.

To further evaluate whether these findings could be generalized to different databases we utilized our Minnesota twins database. The Minnesota Twin and Family Study (MTFS)⁴⁷ is a large, multi-discipline, multi-year study to examine the interaction between genetic and environmental risk factors in the development of adolescent and adult alcoholism and drug abuse. The advantage of the study is that it uses a population based twin ascertainment in which all same sex twins born in the state of Minnesota are identified by public birth records. The recruitment targets 11 and 17 year old twins. They were administered the parent version of the DICA-R (Diagnostic Interview for Children and Adolescents⁷⁵ and the Structured Clinical Interview for DSM-III-R (SCID-R).⁶⁴ Interviews were administered by individuals who have a bachelor's or master's degree in psychology or a related field. Interviewers also complete an intensive course of training that includes didactic instruction, practice interviews, mentoring by an experiences clinical interviewer, and a written examination covering the DSM disorders assessed. All interviews are tape-recorded. Complete interviews are reviewed in a consensus conference by at least two advanced clinical psychology graduate students. Individual symptoms are reviewed, including listening to the audio tapes as needed, to determine whether the behaviors reported by the interviewees were frequent and severe enough to count as a symptom under DSM. In a study of the reliability of the diagnostic and consensus procedures that involved review of clinical material by two independent teams of clinicians. The advantage of this database is that the assessments are performed by standardized, structured instruments, administered by well trained individuals.

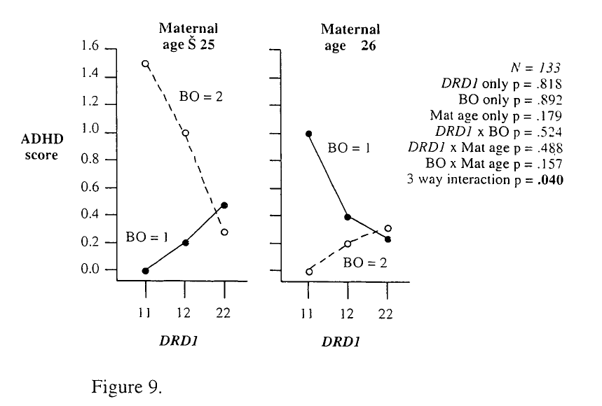

Using the MTFS we examined the role of maternal age and birth order as genostatic factors in the potential association of the DRD1 gene and ADHD (attention deficient hyperactivity disorder). The ADHD score was no DSM diagnosis=0, possible ADHD=1, probably ADHD=2, definite ADHD=3, birth order BO=1, first born, BO=2, second born or greater (See, FIG. 9).

In the probands with maternal age ≦25 and birth order=1, there was a 2 allele codominant association of the DRD1 gene with ADHD. The lowest scores were with the 11 genotype with progressive increases for the 12 and 22 genotypes. By contrast, for those with birth order=2 or more, the highest ADHD scores were associated with the 11 genotype with progressive decreases for the 12 and 22 genotype, i.e, 1 allele codominant. For those with a maternal age of ≧96 years, the effect was reversed. For those with birth order=1, the 11 allele was associated with the highest ADHD score, with progressive decreases across the 12 and 22 genotypes, i.e. 1 allele codominant. For those with a birth order of 2 or more the inheritance was 2 allele codominant.

We have observed the same genostatic effects of maternal age and birth order in four different databases, numerous phenotypes and over 10 different genes. This indicates it is a general phenomena.

### Example VII

Genostasis and the Androgen Receptor (AR) Gene.

The studies of Wang et al⁷³ on maternal age effects in mice suggested that variations in intra-uterine estrogen or androgen levels by maternal age were responsible. We reasoned that if this was the case, genetic variants at the estrogen receptor or androgen receptor gene might also show genostasis effects. An effect of sex hormone genes is also consistent with the fact that many different phenotypes show a marked gender effect. For example the frequency of behavioral phenotypes such as autism, ADHD, ODD, conduct disorder and learning disorders show a 4:1 male to female ratio, while other phenotypes such as depression and obsessive-compulsive disorder show a 2 to 4:1 female to male ratio. Phenotypes such as coronary artery disease, hypertriglyceridemia, rheumatoid arthritis, lupus erythematosis, osteoporosis, Alzheimer's disease, diabetes, and many others show significant gender differences. In addition there are a number of cancers such as breast and prostate that are hormone dependent. These observations suggested to us that various hormone genes might act as genostatic factors.

We found that the estrogen receptor gene had no genostatic effect. By contrast, the AR gene had marked genostatic effects. There are two trinucleotide repeat polymorphisms in exon 1 of the AR gene, CAG³⁶ and GGC,⁶³ resulting in polyamino acid tracts in the protein. When highly expanded 43 to 65 times, the CAG trinucleotide repeat causes X-linked spinal muscular atrophy.⁵³ In the normal population this triplet is repeated 11 to 31 times.³⁶ The GGC⁶³ repeat is less complex and consists predominately of a 16 and a 17 repeat and several minor alleles.

The binding of testosterone to the androgen receptor results in the increased transcription of several AR-responsive reporter genes, a phenomena termed transactivation. The elimination of the CAG tract in both humans and rats, results in increased transcription of the AR gene, suggesting the polyglutamine tract is plays a role in the regulation of the expression of the AR gene.¹¹ Progressive expansion of the CAG and the GGC tract in the human AR gene causes a linear decrease of transactivation function. The reduction of androgen gene expression was proportional to the number of repeats over the range of normal alleles with the shorter alleles showing the greatest activity.¹² The observation that the shorter of both the CAG and GGC alleles are associated with prostate cancer,^{43, 48} an androgen dependent tumor, suggests that the shorter of the normal alleles at both polymorphisms are associated with increased expression of the AR gene and increased transactivation. Since the GGC repeat has fewer alleles it is easier to analyze. We have divided the alleles into two groups, 16 repeats or shorter (≦16) and 17 repeats or longer (≦17). In previous studies we have shown the AR gene is associated with ADHD, CD, ODD and a range of other externalizing behaviors.^{18, 28} We now show that the AR gene can act as an genostatic factor modifying the genotype-phenotype interaction of other genes.

### Example VIII

AR×DRD4 with ODD in the TS Database.

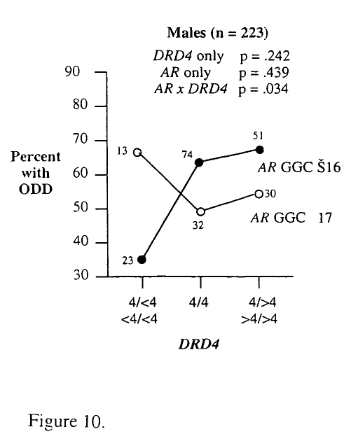

Interest in the potential role of the dopamine D₄ receptor gene (DRD4) in behavioral disorders was stimulated by the report of an association of the DRD4 gene with novelty seeking in two separate studies.^{4, 38} This was replicated in some but not all studies.^{35, 50} The DRD4 gene studies either utilize Cloninger's Tridimensional Personality Inventory¹⁵ which consists of three temperament scales—novelty seeking, harm avoidance, and reward dependence) or the more recent TCI (Temperament Character Inventory^{16, 67} with more extensive assessments. The DRD4 polymorphism is a 48 bp repeat in the second trans-membrane domain.⁷⁰ The alleles consist of 2 to 8 repeats. The 4 repeat allele is most common. The 2 and 7 repeat alleles are next most common and the other alleles are rare. Many studies have examined the presence of the 7 and 8 alleles (eg. 4/7, 4/8, 7/7 genotypes) versus all other genotypes (e.g. 4/4, 2/4 genotypes). In our previous studies²⁶ we have divided the alleles into 3 genotypes consisting of any less than 4 genotypes (4/<4 and <4/<4), 4/4, and any greater than 4 genotype (4/>4 and >4/>4). The two-way interaction between the DRD1 and AR genes with ODD are shown in FIG. 10.

There was a progressive increase in the frequency of comorbid ODD in the TS probands from the 4/<4,<4/<4 DRD4 genotype, to the 4/4 genotype, to the 4/>4, >4/>4 genotypes in the probands carrying the AR GGC≦16 alleles. By contrast, in probands carrying the AR GGC≧17 alleles the highest ODD scores were for those carrying any of the ≦4 repeat DRD4 alleles. The associations with the DRD4 gene alone (p=0.242) and with the AR gene alone (p=0.439) with ODD were not significant while the interaction of the DRD4 and AR genes was significant (p=0.034). This shows that the AR gene can serve as a genostatic factor for the association of the DRD4 gene with ODD.

### Example IX

AR×DRD4 with Novelty Seeking in a College Student and Substance Abuse Database.

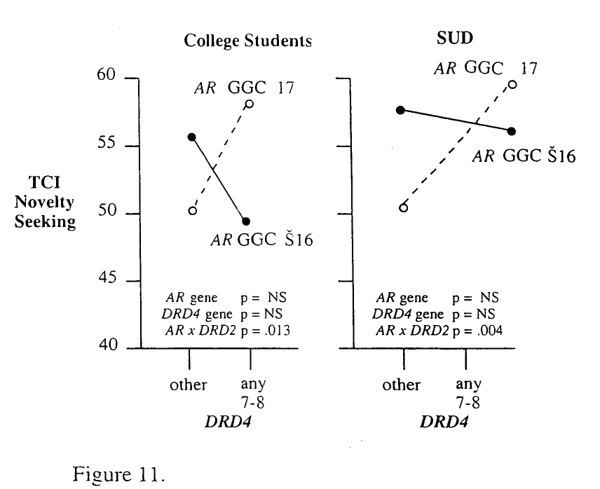

Since most of the studies of novelty seeking and the DRD4 gene were done with the 7+(4/7, 4/8, 7/7 genotypes) versus all others, we examined that scoring using a database involved in studies of genetic factors in substance use disorder (SUD) and a control population of college students. In both databases the subjects had been administered the TCI. These databases have been described elsewhere.²⁴ We examined the college student controls and the SUD subjects separately. These results are shown in FIG. 11.

In both of these independent sets of subjects for those carrying the AR GGC ≦16 alleles, the novelty seeking scores were higher in the DRD4 ‘other’ genotypes than in the genotypes carrying the 7 or 8 allele. By contrast, for those carrying the AR GGC≦17 alleles, the novelty seeking scores were highest for those carrying the DRD4 7 or 8 alleles. This can explain the variability in the DRD4 findings, some studies supporting the original findings while many do not. This is a genostatic effect rather than simply an additive effect of two genes.

### Example X

AR×DRD2 with Novelty Seeking.

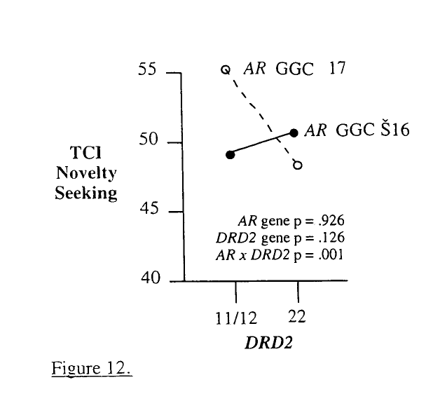

Using the same two databases, we also examined the potential role of the AR gene as a genostatic modifier of the interaction of the DRD2 gene with the TCI novelty seeking score. These results are shown in FIG. 12.

There was an increase in the novelty seeking score from carriers of the DRD2 1 allele to those without the allele (22 genotype) in those TS subjects carrying the AR GGC≦16 allele. By contrast for those carrying the AR≧17 allele, there was a decrease in the novelty seeking score from those carrying the 1 allele to those not carrying this allele. This indicates that the AR gene serves as a genostatic factor for the interaction of the DRD2 gene in novelty seeking.

### Example XI

AR×DRD2 with Depression in the Obesity Database.

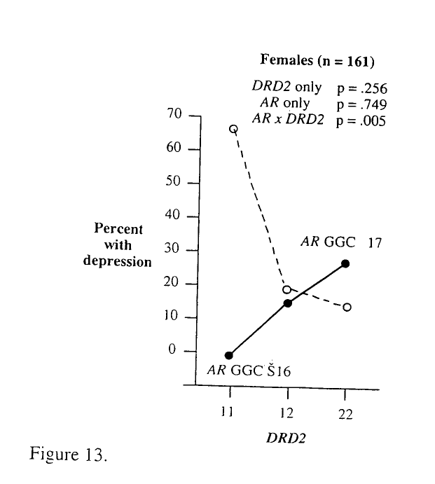

While the DRD2 gene has been repeatedly shown to be associated with a number of externalizing disorders, it has not been associated with internalizing disorders such as depression.⁶² We investigated the possibility that this might be due to the presence of genostatic factors. FIG. 13 shows the two-way interaction of the DRD2 gene Taq I A polymorphism and AR gene with depression in women in the obesity database.

There was a 2 allele codominant association of the DRD2 gene with depression in individuals carrying the AR≦16 alleles. By contrast, in the AR≧17 allele carriers there was a strong 1 allele codominant effect. The association of the DRD2 alone (p=0.256) and the AR gene alone (p=0.749) was negative while the DRD2×AR gene interaction was significant (p=0.005).

### Example XII

AR×HTR2C with Paranoid Personality in the SUD Database.

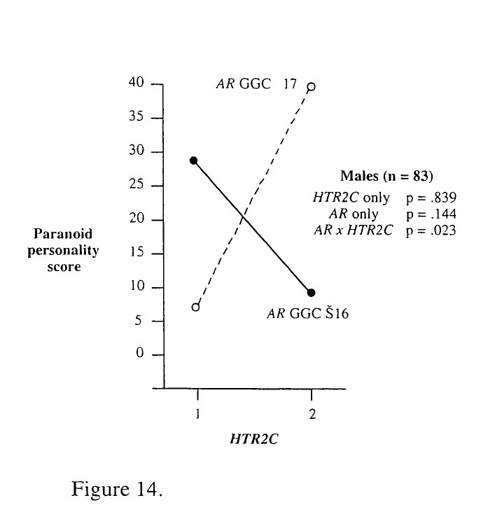

Serotonin has been implicated in many psychiatric and personality disorders. A Cys 23 Ser polymorphism in the HTR2C gene has often been utilized in psychiatric genetics. We examined the role of the AR gene in the association of the HTR2C gene using the Cys 23 Ser polymorphism with the presence or absence of paranoid personality disorder in our SUD database. Since this is an X-linked gene, in males only the 1 and 2 alleles (genotypes) are present. The results are shown in FIG. 14.

In those carrying the AR GGC≦16 alleles, the HTR2C 1 allele was associated with the highest paranoid personality scores. By contrast, those carrying the AR GGC≧17 alleles the 2 allele was associated with the highest score. The HTR2C gene alone and the AR gene alone were not associated with paranoid personality.

### Example XIII

AR×COMT with Tics in the TS Database.

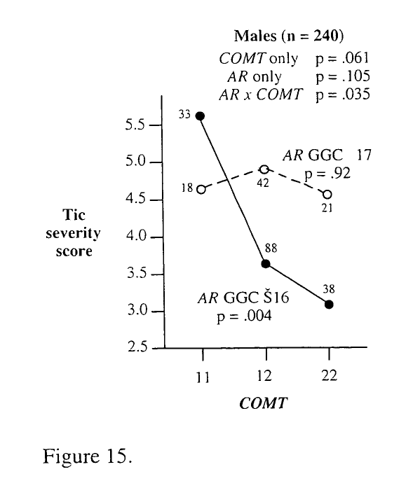

Chronic tics are the main characteristic of TS. Since major neuroleptics with dopamine D₂ receptor antagonist activity decrease tics and stimulants with dopamine agonist properties increase tics, the presence of defects in dopamine metabolism has been one of the major theories of the genetic basis of TS. Since catechol-o-methyl transferse is a major catabolic pathway for dopamine, it has been a major candidate gene for TS. Two studies examining enzyme activity have shown an increase in enzyme activity in TS subjects. This led to studies of a Val 158 Met polymorphism of the COMT gene. This polymorphism is associated with 2 to 4 fold differences in enzyme activity. However, two different studies of the Val 158 Met polymorphism have shown no association with TS.^{3, 10} We examined the possible genostatic effect of the AR gene on the association of the COMT gene with the number of tics in male TS probands. The results are shown in FIG. 15.

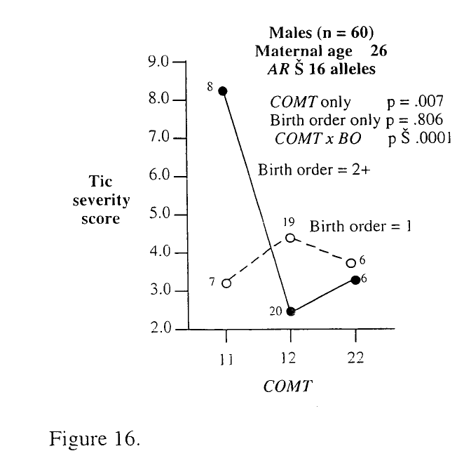

There was only a borderline non-significant interaction of the COMT gene alone with tics. In individuals carrying the AR≦16 alleles 11 (Val/Val) carriers of the COMT variant had the highest tic scores. There was a progressive decrease in the tic score for the 12 and 22 genotypes (p=0.004) consistent with a recessive effect of the Val allele on tics scores. By contrast, for those carrying the AR≧17 alleles, there was no significant change in tic score by COMT genotype (p=0.92). There was no effect of maternal age on the association of the COMT gene with the tic score but among the probands with a maternal age ≧26 and carrying the AR≦16 alleles, there was a strong genostatic effect of birth order. This is shown in FIG. 16.

The greatest correlation between tics and the COMT gene occurred in probands of maternal age ≧26 years, AR≦16 allele and birth order=2 or greater. When the AR gene, maternal age and birth order were not taken into consideration, the r²₀=0.016, p=0.045. When they the were taken into consideration r²₊=0.093, p=0.0001, r²₀/r²₊=5.8 (see below for description of r²₀ and r²₊).

### Example XIV

AR×ADRB2 with Diabetes in the Obesity Database.

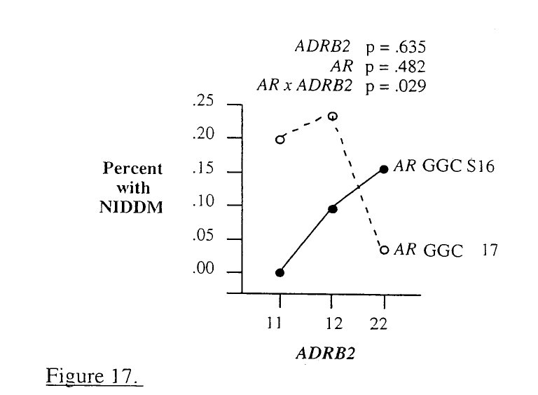

A number of studies have implicated a role of the adrenergic beta 2 receptor gene (ADRB2) in obesity and diabetes.^{30, 37, 52, 69} We utilized a database used for studies of genetic factors in obesity^{6, 27} to examine the possible role of the AR gene as a genostatic factor in diabetes. These results are shown in FIG. 17.

For those who carried the AR GGC≦16 allele, there was a progressive increase in the frequency of NIDDM from 0 percent for those with a ADRB2 genotype of 11, to 10 percent for those with a 12 genotype and 16 percent for those with a 22 genotype. By contrast, for those carrying the AR GGC≧17 allele the frequency of NIDDM was 20 to 24 percent in those carrying the 11 and 12 ADRB2 genotype and decreased to 4 percent in those carrying the 22 genotype. The association of the ADRB2 gene alone, or the AR gene alone with NIDDM, was non-significant while the ADRB2×AR interaction was significant (p=0.029).

### Example XV

AR×11B-HSB1 with Cholesterol in Obesity Database.

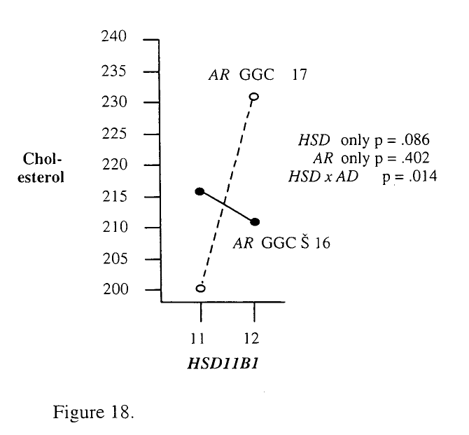

11β-hydroxysteroid dehydrogenase type 1 catalyzes the conversion of active 11-hydroxy glucocorticoids (cortisol) to their inactive 11-keto form (cortisone). It has been implicated as a candidate gene in obesity and the metabolic syndrome.^{55, 65} Since dyslipidemia is one of the major characteristics of the metabolic syndrome, we examined the potential association of a polymorphism of this gene with cholesterol levels in the obesity database. The results are shown in FIG. 18.

There was an opposite association of the 11BHSB1 gene with cholesterol by AR gene alleles. As a result, there was no significant association of the 11BHSB1 gene alone or the AR gene alone but there was a significant two-way interaction between 11BHSB1 and cholesterol (p=0.014).

### Example XVI

Other Genostatic Genes.

We have examined a number of other genes including the estrogen receptor 1 (ESR1), sex binding protein (SBP), aromatase (CYP19), serotonin transporter, and others. To date, only the AR gene as shown this effect.

### Example XVII

Assessing the Increase in Power Due to Genostasis.

We have assessed the increase in power available to genetic studies by including genostatic factors. This was done by comparing the ratio of the r² values with and without genostatic variables. We term this r²₀/r²₊. For example, referring to FIG. 1 (dotted line) the gene scores for the DRD1 gene relevant to OCD would be 11=2, 12=1 and 22=0, for a combined gene score of 210. Using these scores r²=0.003, p=0.379. However, when maternal age is added as a co-factor the gene scores are now 210 for probands with a maternal age of ≧26 years but 012 for those with a maternal age of ≦25. With this scoring r² now=0.026, an 8.7 fold increase in r² or power. Birth order can also be included in the scoring. Since birth order had no effect on gene scoring for the probands with a maternal age of ≦25 years, this scoring was not changed. However, referring to FIG. 7, for those with a maternal age of ≧26 and first born, the DRD1 gene scoring was 200, for the second born it was 220, for the third born it was 021. With this scoring r²=0.042. r²₀/r²₊=14, indicative of a 14 fold increase in power compared to the r² when genostatic factors are not included.

Example XVIII

Implications for Sibling Pair and Linkage Analysis of Complex Disorders.

The tendency for genotype—phenotype associations to vary by birth order has important implications for the power of sibling pair and other methods of linkage analysis. Studies of Risch⁶⁰ have already shown that for genes with a low effect size, association studies have 10 times or more power than sibling pair analysis. The finding that genotype—phenotype associations often reverse themselves across siblings of different birth order, indicates that in practice, compared to sibling pair and lod score analysis, association studies that take genostatic effects into consideration are likely to be even more powerful than the purely mathematical analyses suggest.

Overall, the above examples teach that the involvement of variables, such as maternal age, birth order and the AR gene, are important genostatic factors. When they are not considered, the power to identify the role of specific candidate genes in polygenic disorders is poor. When they are included, power is dramatically increased.

### References

1. Ahima, R. S., Dushay, J., Flier, S. N., Prabakaran, D., & Flier, J. S. Leptin accelerates the onset of puberty in normal female mice. J Clin Invest, 1997; 99:(3), 391-5.

2. Ahima, R. S., Prabakaran, D., Mantzoros, C., Qu, D., Lowell, B., Maratos-Flier, E., & Flier, J. S. Role of leptin in the neuroendocrine response to fasting. Nature, 1996; 382:(6588), 250-2.

3. Barr, C. L., Wigg, K. G., & Sandor, P. Catechol-O-methyltransferase and Gilles de la Tourette syndrome. Mol Psychiatry, 1999; 4:(5), 492-5.

4. Benjamin, J., Li, L., Patterson, C., Greenberg, B. D., Murphy, D. L., & Hamer, D. H. Population and familial association between the D4 dopamine receptor gene and measures of novelty seeking. Nature Genet. 1996; 12: 81-84.

5. Blum, W. F., Englaro, P., Hanitsch, S., Juul, A., Hertel, N. T., Muller, J., Skakkebaek, N. E., Heiman, M. L., Birkett, M., Attanasio, A. M., Kiess, W., & Rascher, W. Plasma leptin levels in healthy children and adolescents: dependence on body mass index, body fat mass, gender, pubertal stage, and testosterone. J Clin Endocrinol Metab, 1997; 82:(9), 2904-10.

6. Bottini, N., MacMurray, J., Peters, W., Rhostamkhani, M., & Comings, D. E. Association of the acid phosphatase (ACP1) gene with triglyceride levels in obese post-menopausal women: potential relevance to the development of metabolic syndrome. (Submitted) 2002.

7. Bouvattier, C., Lahlou, N., Roger, M., & Bougneres, P. Hyperleptinaemia is associated with impaired gonadotrophin response to GNRH during late puberty in obese girls, not boys. Eur J Endocrinol, 1998; 138:(6), 653-8.

8. Butler, M. G., Hedges, L., Hovis, C. L., & Feurer, I. D. Genetic variants of the human obesity (OB) gene in subjects with and without Prader-Willi syndrome: comparison with body mass index and weight. Clin Genet, 1998; 54:(5), 385-93.

9. Carlsson, B., Ankarberg, C., Rosberg, S., Norjavaara, E., Albertsson-Wikland, K., & Carlsson, L. M. Serum leptin concentrations in relation to pubertal development. Arch Dis Child, 1997; 77:(5), 396-400.

10. Cavallini, M. C., Di Bella, D., Catalano, M., & Bellodi, L. An association study between 5-HTTLPR polymorphism, COMT polymorphism, and Tourette's syndrome. Psychiatry Res, 2000; 97:(2-3), 93-100.

11. Chamberlain, N. L., Driver, E. D., & Miesdeld, R. L. The length and location of CAG trinucleotide repeats in the androgen receptor N-terminal domain affect transactivation function. Nucleic Acids Res. 1994; 22: 3181-3186.

12. Choong, C. S., Kemppainen, J. A., Zhou, A. -X., & Wilson, E. M. Reduced androngen receptor gene expression with first exon CAG repeat expansion. Molec.Endocr. 1996; 10: 1527-1535.

13. Clayton, P. E., & Trueman, J. A. Leptin and puberty. Arch Dis Child 2000; 83:(1), 1-4.

14. Clement, K., Vaissa, C., Lahlou, N., Cabrol, S., Pelloux, V., Cassuto, D., Gourmeien, M., Dina, C., Chambaz, J., Lacorte, J. -M., Basdevant, A., Bougneres, P., Lebouc, Y., Frougel, P., & Guy-Grand, B. A mutation in the human lepitin receptor gene causes obesity and pituitary dysfunction. Nature 1998; 392: 398-401.

15. Cloninger, C. R., Przybeck, T. R., & Svrakic, D. M. The Tridimensional Personality Questionnaire. U.S. Normative data. Psychol.Rep. 1991; 69: 1047-1057.

16. Cloninger, C. R., Svrakic, D. M., & Przybeck, T. R. A psychobiological model of temperament and character. Arch. Gen.Psychiatry 1993; 50: 975-990.

17. Comings, D. E. Polygenic Inheritance. Nature Encyclopedia of the Genome (2002). MacMillian.

18. Comings, D. E., Chen, C., Wu, S., & Muhleman, D. Association of the androgen receptor gene (AR) with ADHD and conduct disorder. Neuroreport 1999; 10: 1589-1592.

19. Comings, D. E., & Comings, B. G. A controlled study of Tourette syndrome. I-VII. Am.J.Hum. Genet. 1987; 41: 701-866.

20. Comings, D. E., & Comings, B. G. Comorbid Behavioral Disorders. R. Kurlan Handbook of Tourette's Syndrome and Related Tic and Behavioral Disorders (1993. pp. 111-147). New York: Marcel-Decker.

21. Comings, D. E., Gade-Andavolu, R., Gonzalez, N., Wu, S., Lesieur, H. R., Rugle, L. J., & Rosenthal, R. J. The additive effect of neurotransmitter genes in pathological gambling. The interaction of 31 candidate genes. Clinical Genetics 2001;60: 107-116.

22. Comings, D. E., Gade-Andavolu, R., Gonzalez, N., Wu, S., Muhleman, D., Blake, H., Chiu, F., Wang, E., Farwell, K., Darakjy, S., Baker, R., Dietz, G., Saucier, G., & MacMurray, J. P. Multivariate analysis of associations of 42 genes in ADHD, ODD and conduct disorder. Clin Genet 2000; 58:(1), 31-40.

23. Comings, D. E., Gade-Andavolu, R., Gonzalez, N., Wu, S., Muhleman, D., Blake, H., Dietz, G., Saucier, G., & MacMurray, J. P. Comparison of the role of dopamine, serotonin, and noradrenaline genes in ADHD, ODD and conduct disorder: multivariate regression analysis of 20 genes. Clin Genet 2000; 57: 178-96.

24. Comings, D. E., Gade-Andavolu, R., Gonzalez, N., Wu, S., Muhleman, D., Blake, H., Mann, M. B., Dietz, G., Saucier, G., & MacMurray, J. P. A multivariate analysis of 59 candidate genes in personality traits: the temperament and character inventory. Clin Genet 2000; 58:(5), 375-85.

25. Comings, D. E., Gade, R., Muhleman, D., Peters, W. R., & MacMurray, J. P. The LEP gene and Age of Menarche: Maternal age as a Potential Cause of Hidden Stratification in Association Studies. Molecular Genetics and Metabolism 2001; 73: 204-210.

26. Comings, D. E., Gonzalez, N., Wu, S., Gade, R., Muhleman, D., Saucier, G., Johnson, P., Verde, R., Rosenthal, R. J., Lesieur, H. R., Rugle, L. J., Miller, W. R., & MacMurray, J. P. Studies of the 48 bp repeat of the DRD4 gene in impulsive-addictive behaviors: Tourette syndrome, ADHD, pathological gambling, and substance abuse. Am.J.Med.Gen. (Neuropsych. Genet.) 1999; 88: 358-368.

27. Comings, D. E., MacMurray, J. P., Gade, R., Muhleman, D., & Peters, W. R. Genetic variants of the human obesity gene: Association with psychiatric symptoms and body mass index in young women, and interaction with the dopamine D2 receptor gene. Molecular Psychiatry 1996; 1: 325-335.

28. Comings, D. E., Muhleman, D., Johnson, J. P., & MacMurray, J. P. Parent-Daughter transmission of the Androgen Receptor (AR) gene as an explanation of the effect of father absence on age of menarche. Child Development 2002; (in press).

29. Comings, D. E. R. M. M. D. D. G. C. L. -C. M. J. Multi-gene Tests with ROC Plots for the Assessment of Risk for Polygenic Disorders: Late Onset Alzheimer's Disease as an Example. (in press).

30. Corbalan, M. S., Marti, A., Forga, L., Martinez-Gonzalez, M. A., & Martinez, J. A. beta(2)-Adrenergic receptor mutation and abdominal obesity risk: Effect modification by gender and HDL-cholesterol. Eur J Nutr, 2002; 41:(3), 114-8.

31. Daly, M. J., Rioux, J. D., Schaffner, S. F., Hudson, T. J., & Lander, E. S. High resolution haplotype structures in the human genome. Nature Genetics 2001; 29: 229-232.

32. Devlin, B., & Roeder, K. Genomic control for association studies. Biometrics, 1999; 55:(4), 997-1004.

33. Devlin, B., Roeder, K., & Bacanu, S. -A. Unbiased methods for population-based association studies. Genetic Epidemiology 2001; 21: 273-284.

34. Drago, F., Contarino, A., & Busa, L. The expression of neuropeptide-induced excessive grooming behavior in dopamine D1 and D2 receptor-deficient mice. Eur J Pharmacol, 1999; 365:(2-3), 125-31.

35. Ebstein, R. P., & Belmaker, R. H. Saga of an adventure gene: Novelty seeking, substance abuse and the dopamine D4 receptor (DRD4) exon III repeat polymorphism. Molecular Psychiatry 1997; 2: 381-384.

36. Edwards, A., Hammond, H. A., Jin, L., Caskey, C. T., & Chakraborty, R. Genetic variation at five trimeric and tetrameric tandem repeat loci in four human population groups. Genomics 1992; 12: 241-253.

37. Ellsworth, D. L., Coady, S. A., Chen, W., Srinivasan, S. R., Elkasabany, A., Gustat, J., Boerwinkle, E., & Berenson, G. S. Influence of the beta2-adrenergic receptor Arg16Gly polymorphism on longitudinal changes in obesity from childhood through young adulthood in a biracial cohort: the Bogalusa Heart Study. Int J Obes Relat Metab Disord, 2002; 26:(7), 928-37.

38. Epstein, R. P., Novick, O., Umansky, R., Priel, B., Osher, Y., Blaine, D., Bennett, E. R., Nemanov, L., Katz, M., & Belmaker, R. H. Dopamine D4 receptor (D4DR) exon III polymorphism associated with the human personality trait of novelty seeking. Nature Genet. 1996; 12: 78-80.

39. Fowler, J. S., Volkow, N. D., Wang, G. J., Pappas, N., Logan, J., Shea, C., Alexoff, D., MacGregor, R. R., Schlyer, D. J., Zezulkova, I., & Wolf, A. P. Brain monoamine oxidase A inhibition in cigarette smokers. Proc Natl Acad Sci U S A, 1996; 93:(24), 14065-9.

40. Freeman, R. D., Fast, D. K., Burd, L., Kerbeshian, J., Robertson, M. M., & Sander, P. An international perspective on Tourette syndrome: selected findings from 3,500 individuals in 22 countries. Developmental Medicine and Child Neurology 2000; 42: 436-447.

41. Frisch, R. E., & McArthur, J. W. Menstrual cycles: fatness as a determinant of minimum weight for height necessary for their maintenance or onset. Science, 1974; 185:(4155), 949-51.

42. Garcia-Mayor, R. V., Andrade, M. A., Rios, M., Lage, M., Dieguez, C., & Casanueva, F. F. Serum leptin levels in normal children: relationship to age, gender, body mass index, pituitary-gonadal hormones, and pubertal stage. J.Clin.Endocrinol.Metab. 1997; 82: 2849-2855.

43. Giovannucci, E., Stampfer, M. J., Krithivas, K., Brown, M., Brufsky, A., Hennekens, C. H., & Kantoff, P. W. The CAG repeat within the androgen receptor gene and its relationship to prostate cancer. Proc.Natl.Acad.Sci. U.S.A. 1997; 94: 3320-3323.

44. Green, E. D., Maffei, M., Braden, V. V., Proenca, R., DeSilva, U., Zhang, Y., Chua, S. C. J., Leibel, R. L., Weissenbach, J., & Friedman, J. M. The human obese (OB) gene: RNA expression pattern and mapping on the physical, cytogenetic, and genetic maps of chromosome 7. Genome Research 1995; 5: 5-12.

45. Hanley, J. A., & McNeil, B. J. The meaning and use of the area under a receiver operating characteristic (ROC) curve. Radiology 1982; 143: 29-36.

46. Holliday, R. The inheritance of epigenetic defects. Science, 1987; 238:(4824), 163-70.

47. Iacono, W. G., Carlson, S. R., Taylor, J., Elkins, I. J., & McGue, M. Behavioral disinhibition and the development of substance-use disorders: Findings from the Minnesota Twin Family Study. Development and Psychopathology 1999; 11: 869-900.

48. Irvine, R. A., Yu, M. C., Ross, R. K., & Coetzee, G. A. The CAG and GGC microsatellites of the androgen receptor gene are in linkage disequilibrium in men with prostate cancer. Cancer.Res. 1995; 55: 1937-1940.

49. Jeffreys, A. J., Kauppi, L., & Neumann, R. Intensely punctate meiotic recombination in the class II regtion of the major histocompatibility complex. Nature Genetics 2001; 29: 217-222.

50. Jönsson, E. G., Nöthon, M. M., Gustavsson, j. P., Neidt, H., Forslund, K., Matila-Evenden, M., Rylander, G., Propping, P., & Åsberg, M. Lack of association between dopamine D4 receptor gene and personality traits. Psychological Medicine 1998; 28: 985-989.

51. Kiess, W., Muller, G., Galler, A., Reich, A., Deutscher, J., Klammt, J., & Kratzsch, J. Body fat mass, leptin and puberty. J Pediatr Endocrinol Metab 2000; 13 Suppl 1: 717-22.

52. Kim, S. H., Kim, D. J., Seo, I. A., Min, Y. K., Lee, M. S., Kim, K. W., & Lee, M. K. Significance of beta2-adrenergic receptor gene polymorphism in obesity and type 2 diabetes mellitus in Korean subjects. Metabolism, 2002; 51:(7), 833-7.

53. La Spada, A. R., Wilson, E. M., Lubahn, D. B., Harding, A. E., Fischbeck, K. H., Clark, C., Kelly, K. F., Smith, N., Fairweather, N., Brown, T., Johnston, A., & Haites, N. E. Androgen receptor gene mutations in X-linked spinal and bulbar muscular atrophy. Prenatal diagnosis for dystrophia myotonica using the polymerase chain reaction. Nature 1991; 11: 467-470.

54. Mantzoros, C. S. Role of leptin in reproduction. Ann N Y Acad Sci, 2000; 900: 174-83.

55. Masuzaki, H., Paterson, J., Shinyama, H., Morton, N. M., Mullins, J. J., Seckl, J. R., & Flier, J. S. A transgenic model of visceral obesity and the metabolic syndrome. Science, 2001; 294:(5549), 2166-70.

56. Metz, C. E. Basic principles of ROC analysis. Seminars in Nuclear Medicine 1978; 8: 283-298.

57. Murphy, D. L. The Serotonin Connection in OCD—Comments on ‘Recent Advances in Obsessive-Compulsive Disorder’. Neuropsychopharmacol. 1991; 5: 11-12.

58. Reich D E and Goldstein D B. Detecting association in a case-control study while correcting for population stratification. Genet Epidemiol 2001; 20:: 4-16.

59. Risch, N., & Merikangas, K. The future of genetic studies of complex human diseases. Science 1996; 273: 1516-1517.

60. Risch, N. J. Searching for genetic determinants in the new millennium. Nature, 2000; 405:(6788), 847-56.

61. Seki, T., Tanaka, T., & Nakamura, Y. Genomic structure and multiple single-nucleotide polymorphisms (SNPs) of the thiopurine S-methyltransferase (TPMT) gene. Journal of Human Genetics, 2000; 45:(5), 299-302.

62. Serretti, A., & Smeraldi, E. Dopamine D2 receptor gene not associated with symptomatology of mood disorders. Am J Med Genet, 1999; 88:(4), 294-7.

63. Sleddens, H. F., Oostra, B. A., Brinkman, A. O., & Trapman, J. Trinucleotide (GGN) repeat polymorphism in the human androgen receptor (AR) gene. Hum.Molec.Genet. 1993; 2: 493.

64. Spitzer, R. L., Williams, J. B. W., & Gibbon, M. Structured Clinical Interview for DSM-III-R1987. New York: Elsevier Science Publishers.

65. Stewart, P. M., & Tomlinson, J. W. Cortisol, 11beta-hydroxysteroid dehydrogenase type 1 and central obesity. Trends Endocrinol Metab, 2002; 13:(3), 94-6.

66. Strobel, A., Issad, T., Camoin, L., Ozata, M., & Strosberg, A. D. A leptin missense mutation associated with hypogonadism and morbid obesity. Nat Genet, 1998; 18:(3), 213-5.

67. Svrakic, D. M., Whitehead, C., Przybeck, T. R., & Cloninger, C. R. Differential diagnosis of personality disorders by the seven-factor model of temperament and character. Arch.Gen.Psychiatry 1993; 50: 991-999.

68. Uitterlinden, A. G., Weel, A. E., Burger, H., Fang, Y., van Duijn, C. M., Hofman, A., van Leeuwen, J. P., & Pols, H. A. Interaction between the vitamin D receptor gene and collagen type Ialpha1 gene in susceptibility for fracture. J Bone Miner Res, 2001; 16:(2),379-85.

69. van Rossum, C. T., Hoebee, B., Seidell, J. C., Bouchard, C., van Baak, M. A., de Groot, C. P., Chagnon, M., de Graaf, C., & Saris, W. H. Genetic factors as predictors of weight gain in young adult Dutch men and women. Int J Obes Relat Metab Disord, 2002; 26:(4), 517-28.

70. Van Tol, H. H. M., Wu, C. M., Guan, H. -C., Ohara, K., Bunzow, J. R., Civelli, O., Kennedy, J., Seeman, P., Niznik, H. B., & Javanovic, V. Multiple dopamine D4 receptor variants in human population. Nature 1992; 358: 149-152.

71. Voeller, H. J., Truica, C. I., & Gelmann, E. P. Beta-catenin mutations in human prostate cancer. Cancer Res, 1998; 58:(12), 2520-3.

72. vom Saal, F. S., Timms, B. G., Montano, M. M., Palanza, P., Thayer, K. A., Nagel, S. C., Dhar, M. D., Ganjam, V. K., Parmigiani, S., & Welshons, W. V. Prostate enlargement in mice due to fetal exposure to low doses of estradiol or diethylstilbestrol and opposite effects at high doses. Proc Natl Acad Sci U S A, 1997; 94:(5), 2056-61.

73. Wang, M. -H., & vom Sall, F. S. Maternal age and traits in offspring. Nature 2000; 407: 469-470.

74. Weimann, E., Blum, W. F., Witzel, C., Schwidergall, S., & Bohles, H. J. Hypoleptinemia in female and male elite gymnasts. Eur J Clin Invest, 1999; 29:(10), 853-60.

75. Welner, Z., Reich, W., Heijanic, B., Jung, K., & Amado, H. Reliability, validity, and parent-child agreement studies of the Diagnostic Interview for Children and Adolescents (DICA). J.Amer.Acad.Child Adolescent Psychiat. 1987; 26: 649-653.

76. Zhang, S., & Zhai, H. Quantitative similarity-based association tests using population samples. Am.J.Hum. Genet. 2001; 69: 601-604.

77. Zweig, M. H., & Campbell, G. Receiver-operating characteristic (ROC) plots: a fundamental evaluation tool in medicine. Clinical Chemistry 1993; 39: 561-577.

Papers and patents listed in the disclosure are expressly incorporated by reference in their entirety. It is to be understood that the description, specific examples, and figures, while indicating preferred embodiments, are given by way of illustration and exemplification and are not intended to limit the scope of the present invention. Various changes and modifications within the present invention will become apparent to the skilled artisan from the disclosure contained herein. Therefore, the spirit and scope of the appended claims should not be limited to the description of the preferred versions contained herein.

1. A method for identifying a genostatic factor in a polygenic disorder comprises the steps of:
a) identifying the genotypes of a polygene;
b) choosing a variable;
c) performing a statistical analysis between the variable and the genotypes; and
d) determining the variable to be the genostatic factor in the presence of statistical significance.

2. The method of claim 1 wherein the genostatic factor is selected from the group consisting of maternal age, birth order, an AR gene, age and gender.

3. The method of claim 1 wherein the statistical analysis is performed using a statistical method selected from the group consisting of hierarchical ANOVA, multivariate regression analysis, multivariate logistic regression analysis, and three way chi square analysis.

4. A method of determining the association between a plurality of genes and a polygenic disorder comprises the steps of:
a) identifying the genotypes of each gene;
b) choosing a genostatic factor;
c) analyzing a genostatic effect on genotypes and
d) determining the gene to be a polygene if the genostatic effect is statistically significant.

5. The method of claim 4 wherein the genostatic factor is selected from the group consisting of maternal age, birth order, an AR gene, age and gender.

6. The method of claim 4 wherein the genostatic effect is analyzed using a statistical method selected from the group consisting of hierarchical ANOVA, multivariate regression analysis, multivariate logistic regression analysis, and three way chi square analysis.

7. The method of claim 6 wherein the statistical method is performed using a computer program.

8. The method of claim 6 further comprising a step of analyzing the epistatic effect of the genes.

9. A method of analyzing a genostatic effect of a genostatic factor on a plurality of polygenes comprising the step of performing a statistical analysis between the polygenes and a genostatic factor.

10. The method of claim 9 wherein the genostatic factor is selected from the group consisting of maternal age, birth order, an AR gene, age and gender.

11. The method of claim 9 wherein the statistical analysis is selected from the group consisting of hierarchical ANOVA, multivariate regression analysis, multivariate logistic regression analysis, and three way chi square analysis.

12. The method of claim 9 wherein the statistical analysis is performed using a computer program.

13. A method of assessing the risk of polygenic disorders in the presence of a plurality of polygenes comprising the steps of:
a) performing a statistical analysis for genostatic effect of a plurality of genostatic factors on each polygene;
b) choosing the polygene if the genostatic effect is statistical significant;
c) scoring the genotypes of the polygene of (b) in the presence of the genostatic factors,
d) calculating the total variance of a plurality of polygenes of (c);
e) retaining polygenes of (d) with statistical effect;
f) computing a composite risk score of all the polygenes of (e); and
h) evaluating the sensitivity and specificity of the risk of the polygenic disorders.

14. The method of claim 13 wherein the genostatic factor is selected from the group consisting of maternal age, birth order, an AR gene, age and gender.

15. The method of claim 13 wherein the statistical analysis is selected from the group consisting of hierarchical ANOVA, multivariate regression analysis, multivariate logistic regression analysis, and three way chi square analysis.

16. The method of claim 13 wherein the sensitivity and specificity of the risk is evaluated by plotting the composite risk cores in a Receiver Operator Characteristic plot.

17. A method of determining whether a genotype is associated with a polygenic disorder comprising:
a) obtaining information about genostatic factors present in the individuals from whom the genetic material was obtained;
b) performing a first statistical analysis of the association of the genotype with the polygenic disorder
c) processing the data obtained in step (b) to further perform a second statistical analysis using genostatic factors as variables; and
d) evaluating the statistical analysis obtained in step (c).

18. The method of claim 17 wherein the genostatic factors are selected from the group consisting of maternal age, birth order, an AR gene, age and gender.

19. The method of claim 17 wherein the statistical analysis is selected from the group consisting of hierarchical ANOVA, multivariate regression analysis, multivariate logistic regression analysis, and three way chi square analysis.

20. A computer program product comprising a computer memory having a computer software program, wherein the computer software program when executed by a computer processor performs the statistical analysis of claims 1, 9, 13 and 17.
